(19) United States
(12) Patent Application Publication
(10) Pub. No.: US 2012/0202701 A1
(43) Pub. Date: Aug. 9, 2012

(54) AN ANTIBODY-GLYCAN COMPLEX TARGETING THE DISIALYL CORE II AND SIALYL LEWIS X STRUCTURES, AND USES THEREOF INVOLVING ANALYSIS OF STEM CELLS OR CANCER CELLS

(75) Inventors: Suvi Natunen, Vantaa (FI); Johanna Nystedt, Helsinki (FI); Heidi Anderson, Helsinki (FI); Jari Natunen, Vantaa (FI)

(73) Assignees: GLYKOS FINLAND OY, Helsinki (FI); SUOMEN PUNAINEN RISTI, VERIPALVELU, Helsinki (FI)

(21) Appl. No.: 13/265,953

(22) PCT Filed: Apr. 23, 2010
(86) PCT No.: PCT/FI10/50338

§ 371 Date: Nov. 15, 2011

(30) Foreign Application Priority Data
Apr. 23, 2009 (FI) 20095452

(51) Int. Cl. G01N 33/53; C40B 30/04; C08B 37/00; C07K 16/00

(52) U.S. Cl. 506/9; 530/391.1; 536/123.1; 435/7.1

(57) ABSTRACT

Antibody-saccharide-complexes and methods and uses related to analysis of cells. Also disclosed is a method of selection of new antibody with CHO-specificity and to a use of antibodies produced for the analysis of stem cells or cancer cells or other cells or tissues known to bind to CHO-antibodies.

### BACKGROUND

CHO-131 Antibody

CHO-131 is a monoclonal antibody against a sialylated O-glycan glycan epitope. It has been raised against CHO cells transfected with α1,3-fucosyltransferase VII, core 2 N-acetylglucosaminyltransferase and P-selectin glycoprotein ligand 1 to generate an antibody against selectin ligand oligosaccharides (Walcheck et al. 2002, Blood 99(11) 4063-4069). mAb CHO-131 has been shown to bind to glycopeptides with a core 2 O-glycan having the sialyl Lewis X epitope on the β1,6-GlcNAc-branch. The antibody does not bind to glycopeptides where the sialyl Lewis x is carried on the β1,3-Gal-branch, or where the epitope on the β1,6-branch lacks fucose (Walcheck 2002). However, the glycopeptides used to determine the specificity of mAb CHO-131 did not have sialic acid on the β1,3-galactose. The present invention revealed additionally novel and unexpected non-fucosylated core II O-glycan specificity CHO-131 antibodies, when the β1,3-linked galactose is sialylated.

It is realized that the antibody and the target structure have been known separately but the invention revealed novel specificity combining an antibody, which would not have been considered to recognize that specific target structure containing the disialylated non-fucosylated core II structure. This allows novel analysis methods.

The antibodies are useful for the characterization of e.g. mesenchymal stem cells or differentiated mesenchymal stem cells, or hematopoietic stem cells. The invention is especially directed to development of antibody specificity for effective recognition of the non-fucosylated structure and recognition of majority of stem cells by the antibody. The present invention allows optimization of the core II O-glycan recognition and production of new antibodies useful for characterization of cells, especially stem cells.

### SUMMARY OF THE INVENTION

The present invention revealed a novel and unexpected non-fucosylated core II O-glycan specificity CHO-131 antibodies. It is now revealed that the structure is especially useful stem cell marker and that the novel specificity is especially useful for the analysis of stem cells containing the structure. Furthermore the invention is directed to optimization of CHO-131 antibody specificity more effective recognition of non-fucosylated sialylated core II structures.

The invention describes a novel complex of an antibody and the sialylated core II glycan. The present invention describes novel assay of antibody using selected glycan structures and specific binding efficacies to these to reveal novel useful antibody specificity. The invention is further directed to specific glycan array in complex with an antibody, wherein the antibody binding the preferred target structure according to the invention and optionally other CHO-131 targets and does not bind defined control structures.

The invention is directed to use of the novel specificity for selection and analysis of novel CHO-131-antibodies with the novel specificity for recognition of non-fucosylated core II structures. The invention is especially directed to development of antibody specificity for effective recognition of the non-fucosylated structure and recognition of majority of stem cells or differentiated mesenchymal cells from a mesenchymal stem cell culture by the antibody or recognition of majority hematopoietic stem cells from a stem cell containing preparation such as human cord blood hematopoietic stem cell preparation.

The present method would improve selection of new reagents and validation of assays of mesenchymal stem cells or osteogenically or adipocyte differentiated mesenchymal stem cells as described in PCT/FI 2008/050019 and hematopoietic stem cells as described in PCT/FI 2008/050017.

The invention is directed to validation methods for antibodies recognizing effectively the non-fucosylated disialyl core II structures.

### BRIEF DESCRIPTION OF THE DRAWINGS

FIG. 1. Glycan binding specificity of mAb CHO-131. Schematic representations of the glycan structures are shown for the best binders. Symbols: diamond: N-acetylneuraminic acid, circle: galactose, dark square: N-acetylglucosamine, light square: N-acetylgalactosamine, triangle: fucose, SO₃: sulphate, horizontal line: β1,4-linkage, rising diagonal line: β1,3-linkage (for sialic acid α2,3-linkage), declining diagonal line: β1,6-linkage.

FIG. 2. Glycan binding specificity of mAb CSLEX. Schematic representations of the glycan structures are shown for the best binders. Symbols: diamond: N-acetylneuraminic acid, circle: galactose, dark square: N-acetylglucosamine, light square: N-acetylgalactosamine, triangle: fucose, SO₃: sulphate, horizontal line: β1,4-linkage, rising diagonal line: β1,3-linkage (for sialic acid α2,3-linkage), declining diagonal line: β1,6-linkage.

FIG. 3. Labeling of CD34+ (light) and CD34− (dark) cord blood hematopoietic cells by the sialyl Lewis x mAb:s CSLEX, KM93 and CHO-131. The columns represent the mean of three different experiments where mononuclear cells from three different cord blood units were used.

### DETAILED DESCRIPTION OF THE INVENTION

Sialylated Core 2 O-Glycan as a Marker for Hematopoietic Stem Cells, Mesenchymal Stem Cells, and Cell Types Differentiated from Mesenchymal Stem Cells

Our results show that CHO-131 retains binding activity to a non-fucosylated structure, when the β1,3-galactose is substituted by α2,3-sialic acid [Neu5Acα2-3Galβ1-3(Neu5Acα2-3Galβ1-4GlcNAβ1-6)GalNAcα]. This novel binding epitope for CHO-131 explains the differential labeling of cells by CHO-131 and the anti-sialyl Lewis x antibody CSLEX. The unexpected specificity of mAb CHO-131 defines a glycan epitope on hematopoietic and mesenchymal stem cells that can be used in the isolation and analysis of stem cells.

It is realized that the antibody and the target structure has been known separately but the invention revealed novel specificity combining an antibody, which would not have been considered to recognize that specific target structure containing the disialylated non-fucosylated core II structure. There is no prior publication of this type of specificity. The invention describes a novel complex of an antibody and the sialylated core II glycan. The present invention describes novel assay of antibody using selected glycan structures and specific binding efficacies to these to reveal novel useful antibody specificity. The invention is further directed to specific glycan array in complex with an antibody, wherein the antibody binds to the preferred target structure according to the invention and optionally other CHO-131 targets and does not bind defined control structures.

Binding in present invention means specific binding recognizing the bound saccharides effectively and essentially not recognizing non-bound saccharides. The binding in a preferred embodiment is measured as a solid phase assay, e.g. as in examples. The essentially non-binding preferably means less than average 50% signal of the signals of the best (preferably average of three or 5 five best in Tables 1 or 2 for the respective antibody specificity) binding saccharides, more preferably less than on average 35%, even more preferably less than 20%, even more preferably less than 10%, and most preferably less than 5%. In a preferred embodiment the invention is directed to optimized, practically exclusive or exclusive binding specificity with non binding signals less than 4% even more preferably less than 3 and most preferably less than 2% of the signals of the best binding saccharides.

The saccharides/glycans/oligosaccharides mean oligosaccharide epitopes described, these are preferably non-reducing end oligosaccharide sequences which are not modified by any monosaccharide structures except optionally from the reducing end. The saccharides are in a specific embodiment optionally modified by a chemical derivative smaller than monosaccharides to hydroxyls, preferably to position 6 of the Gal residue of sLex or sialyl-lactosamine, by sulphate or phosphate residues, such charged sulphate structures are shown in Tables. Sialic acid (SA) is any natural or synthetic sialic acid structure, preferably Neu5Ac. In a specific embodiment none of the hydroxyls except optionally at the reducing end are modified.

CHO- or CHO-131 specificity and CHO- or CHO-131 type antibody refers here to antibody specificity similar to the original CHO-131 antibody and the corresponding antibodies, when directed to novel antibody methods the original CHO-131 antibody is excluded.

Novel Complex of CHO and Disialyl-Core II Epitopes Comprising Glycans for Binding and Inhibition Assay

The invention is directed to a combination or complex of a (preferably new) antibody with the disialyl core II glycan binding specificity, such as CHO-131-type specificity, preferably with optimized binding activity

with the target “disialyl(ated) core II” target saccharide and optionally control monosialylated core II saccahride according to the Formula I
(Neu5Acα2-3)ₚGalβ1-3[Neu5Acα2-3Galβ1-4(Fucα1-3)ₘGlcNAcβ1-6]GalNAc[α]ₙR, wherein p, m and n are integers 0 or 1, independently, and the larger reducing end derivatives and conjugates thereof, R indicates reducing end derivative or conjugate, preferably a spacer such as an alkyl spacer;
when p is 0, the glycan is a control monosialylated core II saccharideConjugate is preferably linked to a synthetic chemical conjugate (not a natural biosynthetic cell structure).

The conjugate is preferably linked to i) a polymer such as carbohydrate, polysaccharide (agarose, cellulose, chitosan, dextran, glycosaminoglycan etc), protein such as albumins, KLH (Keyhole limpet hemocyanin), transferrin, or organic polymer such as polyacrylamide or polyether (e.g. Polyethyleneglycol-derivative) or ii) detectable label such as a fluorescent molecule (fluorescein, Alexa fluor etc.), or selectively non-covalently binding molecule such biotin, or analog or multifluoroalkyl or a nucleotide or an antigen iii) further immobilizable organic molecule such as a lipid including hydrophobic alkyl, and aromatic organic molecules comprising preferably more than 10, even more preferably more than 15 carbon atoms, such as C10-30 alkyl or arylalkyl alcohols or fatty acids or amines iv) conjugate is a spacer linking the glycan epitope, preferably through a spacer, to a solid phase such as a plastic, glass or metal surface including microarray plate/matrix, microtiter plate well, gold surface including surface Plasmon resonance. Preferred spacers include e.g. spacer comprising C1-10 alkyls and arylalkyls, and bifunctional forms of molecules in iii) or spacer of the arrays of examples and published analogous array spacers, bifunctional means comprising at least two conjugateble atom or atom group such as amine, alkohol, carboxylic acid, aldehyde, ketone, hydrazide, amino-oxy, alkylamino-oxy, thiol, maleimide, alkyneand azide. The conjugateble atoms or groups are selected so that counterpart of one conjugateble atom/group is conjugateble to reducing end or reducing end derivative such as Ser/Thr/peptide derivative of the saccharide epitope and one conjugateble atom/group is conjugateble to the solid phase, e.g. by amide, oxime, thiol-malemide, aldehyde/ketone-hydrazide, alkyne-azide product, or ester linkage.

There are numerous published commercial protein and saccharide polymer conjugates and synthesis technologies available. In a preferred embodiment the conjugate is formed by a glycosidic linkage, preferably O-, N-, C- or S-glycosidic bond, more preferably an alfa-glycosidic bond. The preferred conjugate or spacer structure may include an amino acid or peptide epitope such as serine or threonine residue being O-glycosidically alfa-linked to the reducing end of the glycan epitope such as in natural O-glycans.

In a preferred embodiment m is 0 and the target structure is “non-fucosylated disialyl core II”.

The invention revealed that the e.g. “CHO-131 type antibodies”, or “disialyl core II antibodies” or “di-SA core Abs”, can in a preferred embodiment be complexed with or bound to an isolated or synthetic glycan or group of glycans comprising the target structure(s) according to the invention. In preferred embodiment the antibodies with the novel specificity are in complex with a synthetic glycan group or a glycan array comprising preferred binding target and binding or non-binding control structures. The combination substance may in a form of a glycan array device, e.g as indicated in examples. In the combination substance the preferred antibody is combined with or bound to the binding structures.

The invention is especially directed to the novel disialyl core II antibodies complexed with or bound to two or more of the target structures, and optionally combined or not combined with several control structures. The control structure are preferably Sialyl-lactosamine structures, more preferably α3-sialylated type 2 N-acetyllactosamine, SAα3Galβ4GlcNAc, wherein SA is sialic acid, preferably Neu5Ac. Other preferred control structures include monofucosylated Core II sLex and sialyllactosamine SAα3Galβ4GlcNAc is non-fucosylated counterpart of the sLex epitope SAα3Galβ4(Fucα3)GlcNAc(βR), wherein R indicates spacer or oligosaccharide epitope according to the invention. Preferred control structures further include
target “monosialyl(ated) core II” control saccharide(s) according to the Formula II Galβ1-3[Neu5Acα2-3Galβ1-4(Fucα1-3)ₘGlcNAcβ1-6]GalAc[α]ₙR, wherein variables are as defined in Formula I. When m is 0, the the structure is referred as non-fucosylated mono sialyl core II structure and when m is 1 the structure is referred as fucosylated mono sialyl core II structure—

Preferably the di-SA core Ab is complexed with or bound to either of the novel disialyl glycan epitopes of Formula I: a) the epitope wherein m is 1 and the structure is fucosylated, referred as “fucosylated di-SA core II”, or, b) the glycan epitope wherein m is 0, referred as “non-fucosylated di-SA core II”.

Terms “complexed with or bound to” are referred together as “combined with”. The combination is formed in a preferred embodiment on a polymer or polymers or on a solid phase or solid phases. The presence of the combination can be observed by measuring the binding e.g. by enzyme linked immunoassay detecting the antibody or fluorescent label or biotin based method for detecting the antibody. In a preferred embodiment the solid phase assay is suitable for detecting combination of the antibody with several glycan epitopes simultaneously such as a microtiter plate assay or an array such as a glycan array assay shown in the examples.

In a preferred embodiment the di-SA core Ab is combined with the fucosylated di-SA core II and it is not combined with (or it is less effectively combined with) the non-fucosylated di-SA core II. The antibody is preferably further combined with “spacer bound sLex structures” and/or “linear polylactosamine sLex structures”, but not not effectively combined with corresponding non-fucosylated structures nor monosialyl core II structures. A preferred antibody combined the structures is CSLEX resembling antibody. In a preferred embodiment the antibody has essentially the binding epitopes of CSLEX antibody. The binding epitopes are variable regions of the antibody heavy and light chains. It has been revealed previously that CSLEX is a useful antibody for the characterization of stem cells such as hematopoietic or mesenchymal stem cells, preferably cells with high fucosylation levels such as fucosylated cells,preferably in vitro fucosylated variants of mesenchymal stem cells or directly in cell culture differentiated variants there of. The novel combination of CSLEX type antibody with the structures: i) fucosylated di-SA core II, ii) not effectively bound or combined with monosialyl-core structures (p is 0 in Formula I)

iii) the spacer sLex structures and iv) polylactosamine sLex structures, but not effectively combined with the non-fucosylated forms of the structures (i,iii, iv) is useful for characterization of new CSLEX type antibodies and

In a preferred embodiment the di-SA core Ab is combined with the fucosylated di-SA core II. The fucosylated and non-fucosylated structures are preferably combined or bound or complexed with the antibody with similar binding efficacy,

or in another embodiment non-fucosylated is combined less effectively or in another embodiment combined with higher binding efficacy, wherein the binding efficacies when different are preferably sunbstantial. Here the less effective is preferably at least 35% of the more effective, more preferably at least 50% and most preferably at least 60% . In case similar efficacies, the less effective binding is less than 40% more preferably less than 30% and most preferably less than 20% lower than than more the effective. The binding efficacies are preferably measured by solid phase binding assay such as an array assay of the examples or an ELISA assay such as described in WO2009060129. More preferably the antibody is preferably further combined with “spacer bound sLex structures” and/or “linear polylactosamine sLex structures”, but not effectively combined with corresponding non-fucosylated structures. A preferred antibody combined with the structures is CHO-131 resembling antibody. In a preferred embodiment the antibody has essentially the binding epitopes of CHO-131 antibody. The binding epitopes are variable regions of the antibody heavy and light chains. It has been revealed previously that CHO-131 is a useful antibody for the characterization of stem cells such as hematopoietic or mesenchymal stem cells. The novel combination of CHO-131 type antibody with the structures: i) fucosylated and non-fucosylated di-SA core II, and ii) preferably further containing monosialylated core II structures wherein the bound structure is fucosylated and non-bound non-fucosylated and further optionally iii) spacer conjugate sLex structures and iv)polylactosamine sLex structures, but optionally not effectively combined with the non-fucosylated forms of the structures (ii-iv) are useful for characterization of new CHO-131 type antibodies.

Accordingly, the invention is especially directed to a complex of an antibody and an isolated glycan comprising a target structure according to Formula 1

Neu5Acα2-3Galβ1-3[Neu5Acα2-3Galβ1-4(Fucα1-3)ₘGlcNAcβ1-6]GalNAc[α]ₙ,

wherein m and n are integers 0 or 1, and the larger reducing end derivatives and conjugates thereof, and wherein said antibody is also capable of binding structure

Neu5Acα2-3Gal β1-4Fucα1-3GlcNAcβ1, and/or

a structure according to Formula 1b

Galβ1-3[Neu5Acα2-3Galβ1-4(Fucα1-3)ₘGlcNAcβ1-6]GalNAc[α]ₙ,

wherein m and n are integers 0 or 1.

Preferably, said antibody in said complex is bound to the structure according to Formula 1, when m is 1, and more preferably said antibody is also capable of binding the structure according to Formula 1, when m is 0. Most preferably, said antibody is also capable of binding the structure according to Formula 1b, when m is 1, but is not essentially capable of binding to the structure according to Formula 1b, when m is 0. Alternatively, said antibody bound to the structure according to Formula 1, when m is 1, is not essentially capable of binding to the structure according to Formula 1, when m is 0. Further, said antibody bound to the structure according to Formula 1, when m is 1, is not preferably capable of binding to the structure(s) according to Formula lb.

Preferably, said complex is in an array of glycan structures, and optionally the array comprises said saccharides the antibody is capable of binding and optionally further said saccharides the antibody is not capable of binding. Said glycan array can be a solid phase conjugated saccharide array.

The invention further revealed several non-binding control glycans, preferably as listed in Tables 1 and 2.

The invention is directed to binding assays such as solid phase assays involving binding of the the antibody the glycan or chemical synthetic conjugate of the glycan. The invention is especially directed to the method for detection of the novel disialyl Core II glycan combination(s) with antibodies with preferred disialyl Core II glycan detecting antibody.

Preferably the solid phase assay or liquid phase assay involves binding of the antibody the oligosaccharide glycan or chemical synthetic conjugate of the glycan.

It is realized that rational production of the novel disialyl Core II glycan binding antibodies such as CHO131 type-antibodies has been impossible because the exact antigen structures were not known. The antibody producing cells/animal may die or get compromised and there is need to get similar or specificity optimised antibodies with a CHO-antibody specificity. The present invention is in a preferred embodiment directed to the rational production of new antibodies with CHO-specificity. The novel method includes steps of
1) providing a sample comprising at least one antibody (or functional antibody fragment binding to an antigen). In preferred embodiment in form of serum or a phage display library.
2) contacting the sample with a glycan structure comprising terminal non-reducing end target structure according to the formula I, preferably non-fucosylated disialyl target structure
3) measuring the binding of the antibody to the oligosaccharide sequence.
4) optionally contacting the antibody sample with at least one preferred control glycan structure, more preferably with two, three, four of most preferably at lest five control glycan structures.
5) optionally selecting antibody with specific binding to the target structures but low or non-existent binding to specificity control saccharides, or in a specific embodiment selecting antibodies with additionally or specifically corresponding sLex specificity.
6) optionally using an oligosaccharide sequence comprising the terminal non-reducing end target saccharide sequence or being the target oligosaccharide or glycopeptide for the inhibition of the binding of the antibody the oligosaccharide sequence,
7) optionally using enzyme selected from the group of fucosyltransferase, sialyltransferase or α3-sialidase enzymes to optimize or reduce the amount of the antibody target structures on cells.

The invention is especially directed to the use of optimization of the binding activity of a CHO-type antibody using the novel target sacchairde sequence.

Selection of New Disialyl-Core II, or CHO-131 Type, Antibodies

The invention allows selection of optimized antibodies against the identified glycan structure. Furthermore the invention is directed to optimization of disialyl core II antibody such as CHO-131 antibody specificity for more effective recognition of the non-fucosylated disialyl core II structures. The invention is directed to use of the novel specificity for selection and analysis of novel CHO-131-antibodies with the novel specificity for recognition of non-fucosylated core II structures.

Antibody Selection and Production Method

The invention is directed to a method of selection of a new antibody with disialyl core II, preferably CHO131-type-specificity, preferably with optimized binding activity to the target saccharide(s) or control saccharides according to the Formula I

The invention is directed to validation methods for antibodies recognizing effectively the non-fucosylated or fucosylated di-SA core II structures.

In a preferred embodiment the validation methods include enzymatic modification of the target structures to binding and/or non-binding structures by enzymatic modification of the structures on cell surfaces.

The invention is further directed to combinations of the antibodies and the saccharide epitopes of the invention wherein the saccharide (s) are used as soluble oligosaccharides or conjugates optionally to measure the interaction in solution or to inhibit binding of the antibody to a target saccharide and further optionally to control saccharide when saccharides are solid phase conjugated as described by the invention or cell/tissue surface conjugated. Typically 0.001 nM to 1 mM, more preferably 1 nM to 100 microM inhibitor calculated as monovalent saccharide of the invention is used as soluble saccharides preferably as inhibitors.

Validation of Disialyl Core II or CHO-131 Antibody Analysis

In another preferred embodiment a specificity assay involving antibody binding to the fucosylated and/or non-fucosylated disialyl core II oligosaccharide is used for the validation of the antibody binding specificity analysis. In a preferred embodiment the assay includes binding to control cells with controlled levels of the disialylated core II O-glycans.

The invention is especially directed to the use of the target saccharide comprising control material for validation of the analysis of the antibody binding to cells or other biological materials. In a preferred embodiment the control material is purified a oligosaccharide preferably conjugated to a solid surface or to control cells in a solid phase assay or used as a soluble inhibitor or soluble analyst (e.g. labelled conjugate for a fluorescence polarization assay) to validate the binding specificity of the antibody.

In a preferred embodiment the invention is directed to di-SA core II- or CHO-131-antibody analysis kit comprising the preferred sequence comprising glycan or glycoconjugate or a cell sample optimized with the glycan structure expression, preferably for the validation of the analysis of the preferred di-SA core II- or CHO-131 target-structure in cells or tissues.

A preferred validation method include enzymatic modification of the target structures. A preferred validation method include enzymatic modification of the target structures to non-binding structures. Another preferred modification of a target structure is changed to another target structure and measuring the change of binding of antibodies. The invention revealed that stem cells comprising the disialylated core II structure can be modified by fucosylation to another target structure the disialylated sialyl-Lewis x structure. The assay measures also change of non- or less effectively antibody binding monosialylated Galβ1-3(Neu5Acα2-3Galβ1-4GlcNAcβ1-6)GalNAcα to an effectively binding the core II sLex structure Galβ1-3[Neu5Acα2-3Galβ1-4(Fucα3)GlcNAcβ1-6]GalNAcα

Additional Preferred Target Structures for the Antibody

The invention revealed further sialyl-Lewis x type target structures for the CHO-131 antibody. Reaction to these depends on the presence of such ligands on the target cells. In the examples the fucosylation increase after fucosyltransferase reactions does likely in part include the poly-n-acetyllactosamine structures.

The tetrasaccharide sLex, Neu5Acα3Galβ4(Fucα3)GlcNAc, is also a ligand for the antibody when linked to Sp8-spacer (see consortium nomenclature, chart no 244) or with somewhat weaker binding when linked to sp0 spacer (chart no 243), or when linked by β3-linked to Gal like in structures Neu5Acα3Galβ4(Fucα3)GlcNAcβ3Gal (chart no 245 third best ligand structure) and further reducing end elongated polylactosamine structures such as Neu5Acα3Galβ4(Fucα3)GlcNAcβ3Galβ4GlcNAcβ (chart no 246) and Neu5Acα3Galβ4(Fucα3)GlcNAcβ3Galβ4(Fucα3)GlcNAcβ3Galβ4(Fucα3)GlcNAc (chart no 242, the comparision to 246 and reducing end structure with some binding (chart no 337 indicates that the reducing end fucoses support the binding). The invention is directed to the polylactosamine control saccharides comprising non-reducing end terminal epitope Neu5Acα3Galβ4(Fucα3)GlcNAcβ3GalβR, which may be further elongated with polylactosamine structures and/or linked or conjugated to a carrier structrure (polymer/solid phase)

The sLex binding on the core II or polylactosamine structures is specific, because the corresponding non-fucosylated structures or core I O-glycan Neu5Acα3Galβ4(Fucα3)GlcNAcβ3GalNAcα (chart no 374) has weaker or practically no binding activity.

Hyaluronic Acid Fragments

The data revealed some binding of hyaluronic acid disaccharide GlcAβ3GlcNAc, the invention is in a preferred embodiment directed to testing or assaying a CHO-131 type antibody with hyaluronic acid oligomers, especially terminal GlcA comprising oligomers, especially for improving reactions with specific cell types and testing effects of hyaluronidase and/or hyaluronan lyase degradation of the cells to the binding of the antibodies to the cells

Selection of New Antibodies Including Disialyl Core II Specificity for Effective Recognition of Stem Cells and Mesenchymal Cells

The present invention is directed to the use of analysis of present antibody specificity to obtain similar and/or optimized new antibodies. The invention is directed to use of the novel specificity for selection and analysis of novel CHO-131-type antibodies with the novel specificity for recognition of non-fucosylated core II structures.

The invention is especially directed to development of antibody specificity for effective recognition of the disialylated core II structures, more preferably the non-fucosylated structure and recognition of majority of stem cells or differentiated mesenchymal cells.

The antibody is assayed for the glycan binding according to the invention and antibodies with preferred specificity are selected. The novel antibody is not the original CHO-131, but a novel derivative or complex thereof or totally new antibody with the same specificity.

The mesenchymal cells are preferably from a stem mesenchymal cell culture or recognition of majority hematopoietic stem cells or differentiated variants thereof from a stem cell containing preparation such as human cord blood hematopoietic stem cell preparation.

Preferred majority of preferred cells recognized by a novel CHO-131 type antibody is over 80% even more preferably over 90%, even more preferably over 95% and most preferably over 97% of the preferred cells.

General methods for selecting new antibodies: antibody selection by phage display screening has been published in Jylhä S et al., WO/2008/092992, methods to select anti-glycan antibodies by phage display methods in Wang L et al., Mol Immunol. 1997 June; 34(8-9):609-18, and methods to obtain anti-glycan antibody by immunization in Galil and Repik, WO/1995/024924. The mammalian glycan array oligosaccharide codes for mammalian printed array version 4.0 are available from the Consortium for Functional Glycomics e.g. through web page http://www.functionalglycomics.org/static/consortium/resources/resourcecoreh12.shtml.

The following definitions are provided for some terms used in this specification. The terms, “immunoglobulin”, “heavy chain”, “light chain” and “Fab” are used in the same way as in the European Patent Application No. 0125023.

“Antibody” in its various grammatical forms is used herein as a collective noun that refers to a population of immunoglobulin molecules and/or immunologically active portions of immunoglobulin molecules, i.e., molecules that contain an antigen binding site or a paratope. Examples of molecules which are described by the term “antibody” herein include, but are not limited to: single chain Fvs (scFvs), Fab fragments, Fab′ fragments, F(ab′) fragments, disulfide linked Fvs (sdFvs), Fvs, and fragments comprising or alternatively consisting of, either a VL or a VH domain. The immunoglobulin molecules of the invention can be of any type (e.g., IgG, IgE, IgM, IgD, IgA and IgY), or subclass of immunoglobulin molecule. Preferably, an antibody of the invention comprises, or alternatively consists of, a VH domain, VH CDR, VL domain, or VL CDR. In broadest sense the term antibody includes any polypeptide with glycan antigen binding structure, paratope, conformation binding specifically or exclusively the preferred glycan epitopes of the invention. It is realized that these can be engineered using antibody variable domain conformations and/or known sLex binding protein structures.

An “antigen-binding site”, a “paratope”, is the structural portion of an antibody molecule that specifically binds an antigen.

Glycolipid and carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 29).

It is assumed that Gal (galactose), Glc (glucose), GlcNAc (N-acetylglucosamine), GalNAc (N-acetylgalactosamine) and Neu5Ac are of the D-configuration, Fuc of the L-configuration, and all the monosaccharide units in the pyranose form. The amine group is as defined for natural galactos-and glucosamines on the 2-position of GalNAc or GlcNAc.

Glycosidic linkages are shown partly in shorter and partly in longer nomenclature, the linkages of the sialic acid SA/Neu5X-residues α3 and α6 mean the same as α2-3 and α2-6, respectively, and with other monosaccharide residues β1-3, β1-3, β1-4, and β1-6 can be shortened as β3, β3, β4, and β6, respectively. Lactosamine refers to type II N-acetyllactosamine, Galβ4GlcNAc, and/or type I N-acetyllactosamine, Galβ3GlcNAc and sialic acid (SA) is N-acetylneuraminic acid (Neu5Ac) or N-glycolylneuraminic acid (Neu5Gc) or any other natural sialic acid including derivatives of Neu5X. The sialic acid are referred together as NeuNX or Neu5X, wherein preferably X is Ac or Gc, optionally Ac or Gc and additional Ac, or sulfate or lactate on flexible positions —NGc-OH, 7, 8, or 9, or truncation of 8 and/or 9 or carbon leaving terminal 7- or 8-position hydroxyl or aldehyde or derivative. Occasionally Neu5Ac/Gc/X may be referred as NeuNAc/NeuAc/NeuNGc/NeuGc/NeuNX. Term glycan means here broadly oligosaccharide or polysaccharide chains present in human or animal glycoconjugates, especially on glycolipids or glycoproteins.

Antibodies. Known methods are used for the production of antibodies, e.g., any suitable host animal is immunized, antibody is expressed from cloned immunoglobulin cDNAs and/or an antibody library such as phage display library is screened, preferably against the preferred target and control saccharides of the invention e.g. as defined in WO2009060129. Monoclonal antibody preparation include hybridoma techniques (Köhler et al., Nature, 256: 495-497, 1975; Kosbor et al., Immunology Today, 4: 72, 1983; Cole et al., Monoclonal Antibodies and Cancer Therapy, Alan R Liss, Inc., pp. 77-96, 1985), all incorporated herein by reference.

Information on useful binder specifities including lectin and elongated antibody epitopes is available from reviews and monographs such as (Debray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Neatherlands and internet databases such as pubmed/espacenet or antibody databases such as www.glyco.is.ritsumei.ac.jp/epitope/, which list glycan specificities of monoclonal antibodies.

The three dimensional structures of the antibodies are in general known, the exact structure of the preferred antibodies of the invention comprises antigen binding variable domain sequences of heavy chain CDR1-3 and light chain CDR1-3 complementary to the three dimensional structures of the binding saccharide epitopes according to the invention. The data of inventors indicates the antibody binding is dependent on the CDR regions. The structure is defined by the glycan structures which conformations are in general well known. The conformations of the disialyl core II structures and control are available by standard glycan modelling e.g. as described in WO/2005/037187, WO/2001/043751 or based on similar structure e.g. from Sweetdb database, at German Cancer Research Center Heidelberg, Central Spectroscopic Division, Im Neuenheimer Feld 280, 69120 Heidelberg, Germanyand Justus-Liebig University GieBen, Institute of Biochemistry and Endocrinology, Frankfurter Str. 100, 35392 GieBen, Germany, (web access http://www.glycosciences.de/sweetdb/structure/) preferred analogous structures with Galβ4GlcNAcβ6GalNAc and core II epitopes with published 3D coordinates include e.g.: LinucsID 2726 (http://www.glycosciences.de/sweetdb/start.php?action=explore linucsid&linucsid=2726, accessed 23.4.2010) and LinucsID 14049 (http://www.glycosciences.de/sweetdb/start.php?action=explore linucsid&linucsid=1404 9, accessed 23.4.2010). The complementary antibody conformation includes regions recognizing sialic acid residues including preferably i) polar and/or basic amino acids, more at least one preferably Lys or Arg, which form an ion bond or hydrogen bond(s) with the carboxylic acid functional group, ii) polar amino acid residue(s) having hydrogen bonding to at least one hydroxyl group of fucose residue or/an aliphatic or aromatic amino acid residue in van der Waals contact with fucose ring and/or methyl proton, iii) large binding site conformation capable of affinity increasing interaction with both sialic acids and fucose residue. The interactions can be further defined by crystallography or molecular modelling based on antibody and glycan structures or NMR such as STD NMR experiments (Maaheimo H et al. Biochemistry 2000, 12778-88). As an example STD NMR experiments are performed with soluble oligosaccharides or monovalent conjugates as described in the publications. In a preferred embodiment the invention is directed to complex having essentially 3D structures of CHO131 (or in other embodiment CSLEX, or separately other preferred sLex binding antibodies according to Mao S., et al PNAS USA 1999 96, 6953-58) and the disialyl core glycans. In preferred embodiment the invention is directed to antibody complexes giving NMR signals corresponding to interaction defined above and/or giving essentially similar NMR signals in STD NMR or the preferred antibody complexes

A preferred protein structure for engineering optimized binding specificities is sLex recognizing antibody selected from the group CHO-13, CSLEX or sLex binding antibodies described in the publication Mao S., et al PNAS USA 1999 96, 6953-58 such as S6, S7, S8,or S10, or alternatively using other sLex binding protein e.g. SABA protein (Mandavi J et al Science 2002, 297, 573-9) or lectin of Anaplasma phagocytophilum (Yago T et al J Biol Chem 2003 37987-97) are known to bind sLex and useful to design new antibodies according to invention by gene library screening and/or molecular modellling. A preferred complex structure according to the invention include at least part of variable domain structures of monoclonal antibodies CHO131 or in other embodiment CSLEX, or separately other preferred sLex binding antibodies according to Mao S., et al PNAS USA 1999 96, 6953-58.

### EXAMPLES

### Example 1

### Glycan Binding Specificities of the Monoclonal Antibodies CHO-131 and CSLEX

Materials and Methods

mAb CHO-131 was purchased from R&D Systems (MAB996). mAb CSLEX was purchased form BD Biosciences (551344). Glycan microarray analysis was carried out by the Consortium for Functional Glycomics. Glycan microarrays were printed as described (Blixt 2004). Version 4.0 of the printed glycan array was used for analysis. Binding analysis was performed at 50 μg/ml of antibody. Data are reported as average RFU of 6 replicates after removal of highest and lowest values.

Results and Discussion

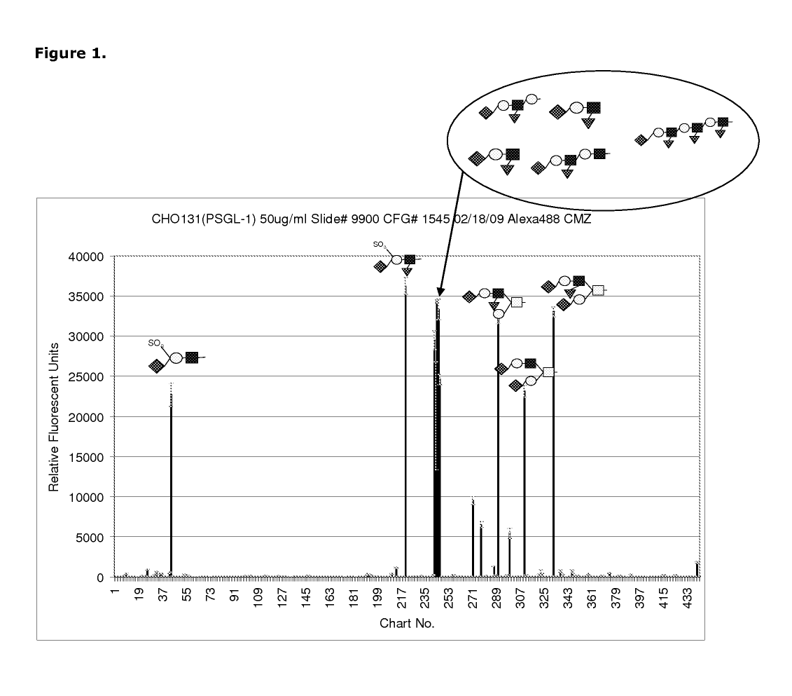

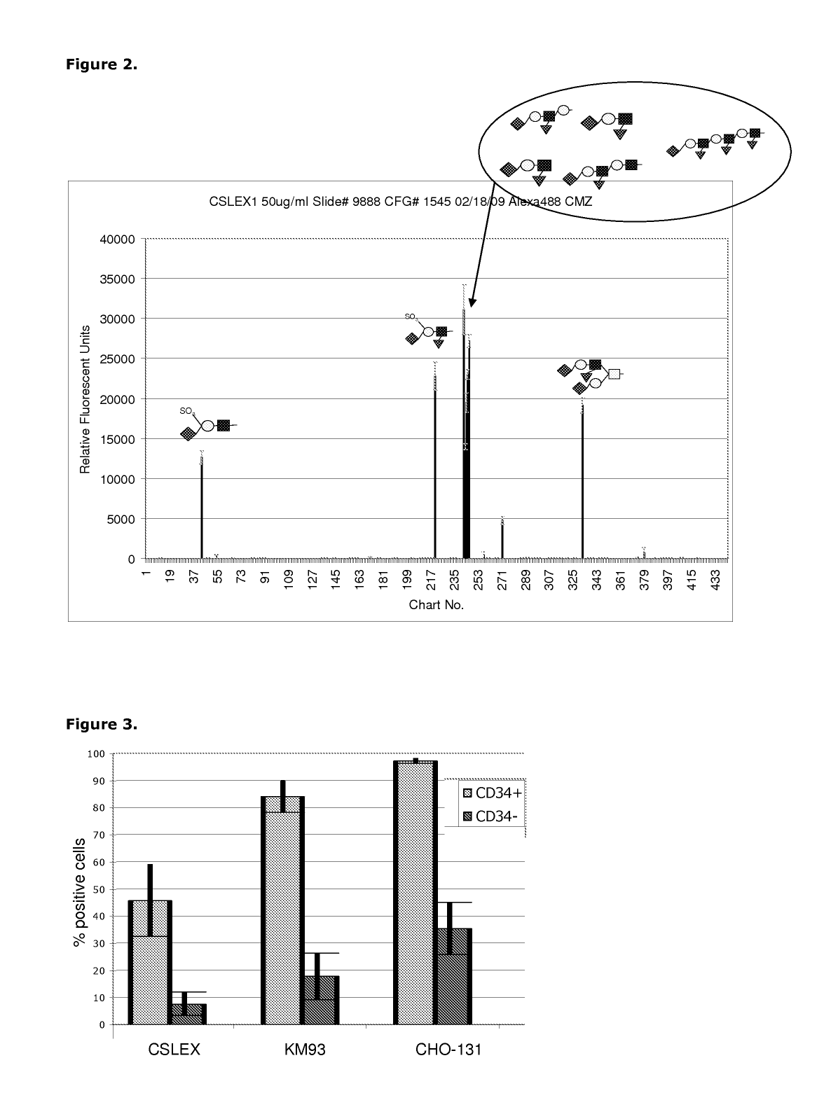

Glycan binding specificity of mAb CHO-131. The glycan binding specificity of mAb CHO-131 on the Consortium for Functional Glycomics glycan microarray is shown in Table 1 and FIG. 1. In addition to the sialyl Lewis x on core 2 0-glycan (chart numbers 332 and 290; RFU 33035 and 32405, respectively), which is the published specificity for mAb CHO-131 (Walchek 2002), mAb CHO-131 bound to several other structures containing the sialyl Lewis x epitope. Notably, a disialylated non-fucosylated core 2 0-glycan structure was a good binder as well (chart number 310; RFU 23122). The same non-fucosylated structure with only one sialic acid bound to CHO-131, but much less efficiently (chart number 277; RFU 6497).

Glycan binding specificity of mAb CSLEX. The glycan binding specificity of the anti-sialyl Lewis x mAb CSLEX on the Consortium for Functional Glycomics glycan microarray is shown in Table 1 and FIG. 1. mAb CSLEX bound to the same sialyl Lewis x containing structures that showed binding to mAb CHO-131, but failed to bind the non-fucosylated core 2 O-glycans (chart numbers 310 and 277; RFU 14 and 0 respectively).

Based on the glycan microarray results, the mAb:s CHO-131 and CSLEX have a similar glycan binding specificity against sialyl Lewis x, with the notable exception that the mAb CHO-131 also binds to non-fucosylated sialylated core 2 O-glycan structures, especially when both of the galactoses are α2,3-sialylated (Neu5Acα2-3Galβ1-3(Neu5Acα2-3Galβ1-4GlcNAcβ1-6)GalNAcα). The difference in the specificities of the two antibodies explains the differential labeling of cells by them (see examples 2-4).

### Example 2

### Recognition of Underfucosylated Cells by CHO-131

Materials and Methods

Bone Marrow (BM) Derived Mesenchymal Stem Cells (MSC:s) BM MSC:s were obtained as described by Leskela et al. (2003). Briefly, bone marrow obtained during orthopaedic surgery was cultured in Minimun Essential Alpha-Medium supplemented with 20 mM HEPES, 10% fetal calf serum, penicillin-streptomycin and 2 mM L-glutamine (all from Gibco). After a cell attachment period of 2 days the cells were washed with PBS, subcultured further by plating the cells at a density of 2000-3000 cells/cm² in the same media and replacing the medium twice a week until near confluence. The cells used in the analyses were of passage 2.

Enzymatic In Vitro α1,3-Fucosylation of BM-Derived MSCs

BM MSCs were detached with 0.25% trypsin/1 mM EDTA in Ca²⁺/Mg²⁺-free PBS (Invitrogen) for 3 minutes. The trypsinization was inhibited by adding excess of αMEM supplemented with 10% human serum albumin (HSA) (Albumin SPR, Sanquin, the Netherlands). Cell viability and cell amounts were determined with trypan blue exclusion. The detached cells were centrifuged 300×g for 5 min, the supernatant was completely removed and 1×106 cells were resuspended in 300 μl enzyme reaction buffer composed of Minimum Essential Medium (MEM) αmedium supplemented with 0.5% HSA (reaction buffer control). 15 mU human recombinant (Spodoptera frugiperda) α1,3-Fucosyltransferase VI (FUTVI) (Calbiochem) and 1 mg GDP-fucose was added per 1×10e6 cells to the reaction buffer for enforced α1,3-fucosylation for 1 hour at +37° C. The original FUTVI enzyme buffer was exchanged to the reaction buffer and activity examined before the experiment. To prevent cell aggregation or cell attachment to the modification vessel, the reactions were resuspended by mechanical pipetting every 30 minutes during the incubation. Parallel reactions with cells only in the reaction buffer without enzymes were always included in each experiment. The enforced enzymatic α1,3-fucosylation was stopped by adding excess reaction buffer and washing the cells twice with reaction buffer. Viability of the α1,3-fucosylated BM-MSCs was monitored by Trypan blue exclusion after which the cells were fixed in 0.5% paraformaldehyde (PFA) before the FACS validation.

FACS Analysis: The Anti-Lex/sLex Glycoform Antibodies Used for FACS Analysis were:

CHO-131, CSLEX (CD15s), HECA 452 and TG-1 (CD15). 1×10⁵ 0.5% PFA fixed BM-MSCs were labelled with 3 μl of unconjugated CHO-131, CSLEX and TG-1 antibodies and 2 μl of FITC-conjugated HECA 452 antibody in Ca²⁺-free PBS supplemented with 0.5% BSA for 15 minutes at room temperature protected from light. Alexa-fluor 488 (Molecular Probes) was used in 1:500 dilution for secondary antibody stainings for the unconjugated primary antibodies for 20 min at room temperature protected from light. The samples were washed in excess PBS+0.5% BSA and analysed with FACSAria (Beckton Dickson) flow cytometer. Analysis was performed using the FACSDiva software (Beckton Dickinson).

Results and Discussion

The staining of bone marrow mesenchymal stem cells with and without enzymatic in vitro fucosylation with antibodies against sialyl Lewis x and Lewis x epitopes is presented in Table 3. The anti-sLex antibodies CSLEX and HECA-452 and the anti-Lex antibody TG-1 stain the native cells only minimally, whereas CHO-131 stains 52% of the native cells. Labeling with all of the three antibodies increases when the cells are enzymatically α1,3-fucosylated. The results indicate, that although all three antibodies recognize fucosylated epitopes, CHO-131 binding is less dependent on fucose than the binding of the other anti-sLex and anti-Lex antibodies. This is consistent with the results presented in Example 1, which show that CH0-131 recognizes both fucosylated (sLex) epitopes and the non-fucosylated structure Neu5Acα2-3Galβ1-3(Neu5Acα2-3Galβ1-4GlcNAcβ1-6)GalNAcα.

### Example 3

### The CHO-131 Epitope is Enriched in CD34+ Hematopoietic Stem Cells

Materials and Methods

Extraction of mononuclear cells (MNCs) from umbilical cord blood. Human term umbilical cord blood (CB) units were collected after delivery with informed consent of the mothers and the CB was processed within 24 hours of the collection. The mononuclear cells (MNCs) were isolated from each CB unit diluting the CB 1:1 with phosphate-buffered saline (PBS) followed by Ficoll-Paque Plus (Amersham Biosciences, Uppsala, Sweden) density gradient centrifugation (400×g/40 min). The mononuclear cell fragment was collected from the gradient and washed twice with PBS.

Depletion of red blood cell precursors by magnetic microbeads conjugated with anti-Glycophorin A (anti-CD235a). MNCs (10⁷) were suspended in 80 μl of 0,5% ultra pure BSA, 2 mM EDTA-PBS buffer. Red blood cell precursors were depleted with magnetic microbeads conjugated with anti-CD235a (Glycophorin a, Miltenyi Biotec) by adding 20 μl of magnetic microbead suspension/10⁷ cells and by incubating for 15 min at 4° C. Cell suspension was washed with 1-2 ml of buffer/10⁷ cells followed by centrifugation at 300×g for 10 min. Cells were resuspended 1,25×10⁸ cells/500 μl of buffer. MACS LD column (Miltenyi Biotec) was placed in a magnetic field and rinsed with 2 ml of buffer. Cell suspension was applied to the column and cells passing through were collected. Column was washed two times with 1 ml of buffer and total effluent was collected. Cells were centrifuged for 10 min at 300×g and resuspended in 10 ml of buffer. All together eight CB units were used for following antibody staining.

Staining with anti-glycan antibodies. Anti-glycan antibodies: mAb CHO-131 was purchased from R&D Systems (MAB996), mAb CSLEX was purchased form BD Biosciences (551344), and mAb KM93 was purchased from Chemicon (MAB2096). MNCs were aliquoted to FACS tubes in a small volume, i.e. 0.5×10⁶ cells/500 μl of 0.3% ultra pure BSA (Sigma), 2 mM EDTA-PBS buffer. Ten microliters of primary antibody was added to cell suspension, vortexed and cells were incubated for 30 min at room temperature. Cells were washed with 2 ml of buffer and centrifuged at 500×g for 5 min. AlexaFluor 488-conjugated anti-mouse (1:500, Invitrogen) secondary antibody was used. Secondary antibodies were diluted in 0.3% ultra pure BSA, 2 mM EDTA-PBS buffer and 200 μl of dilution was added to the cell suspension. Samples were incubated for 30 min at room temperature in the dark. Cells were washed with 2 ml of buffer and centrifuged at 500×g for 5 min. As a negative control cells were incubated without primary antibody and otherwise treated similarly to labeled cells.

Double staining with PE-conjugated anti-CD34-antibody. After staining with anti-glycan antibodies, a double staining with PE-conjugated anti-CD34 antibody (BD Biosciences) was performed. Cells were suspended in 500 μl of buffer and 10 μl of anti-CD34 antibody was added and incubated for 30 min at +4° C. in dark. After incubation cells were washed with 2 ml of buffer and centrifugation at 500×g for 5 min. Supernatant was removed and cells were resuspended in 300 μl of buffer and stored at 4° C. overnight in the dark.

Flow cytometric analysis. The next day cells were analysed with flow cytometer BD FACSAria (BD Biosciences) using FITC and PE detectors. Approximately 250 000-300 000 cells were counted for each anti-glycan antibody. Data was analysed with BD FACSDiva Software version 5.0.2 (BD Biosciences).

Results and Discussion

Results of the double staining experiments with anti-CD34 and three different anti-sialyl Lewis x antibodies are shown in FIG. 3. MAb CHO-131 and Mab KM-93 stained nearly all of CD34-positive cells, whereas MAb CSLEX stained less than half of CD34-positive cells. 10-30% of the CD34 negative cell population was stained with the sLex antibodies.

The differential labeling of CD34+ hematopoietic stem cells by the three anti-sLex antibodies indicates that mAb CHO-131 and mAb KM-93 recognize an epitope on CD34+ cells to which mAb CSLEX does not bind. The glycan binding data presented in Example 1 shows that mAb CHO-131 binds to sialylated non-fucosylated core 2 O-glycan (Neu5Acα2-3Galβ1-3(Neu5Acα2-3Galβ1-4GlcNAcβ1-6)GalNAcα), where as mAb CSLEX requires fucose for binding. Glycan microarray data for mAB KM-93 has been previously prepared by the Consortium for Functional Glycomics. mAb KM-93 and mAb CHO-131 have similar binding specificity toward sLex and the sialylated non-fucosylated core 2 O-glycan structure (www.functionalglycomics.org). The differential labeling of CD34+ cells and the different glycan binding specificities of the antibodies indicate that less than half of CD34+ cells carry the sialyl Lewis x epitope required by mAb CSLEX for binding, but nearly all CD34+ cells carry the sialylated non-fucosylated core 2 O-glycan structure which is recognized by mAbs CHO-131 and KM-93. Both sLex and the sialylated non-fucosylated core 2 O-glycan epitope occur only in a minority of CD34- cells. Therefore binders recognizing the sialylated non-fucosylated core 2 O-glycan epitope could be used to enrich and analyze CD34+ hematopoietic stem cells.

### Example 4

### CHO-131 Epitope in Mesenchymal Stem Cells and Cell Types Differentiated from Mesenchymal Stem Cells

Materials and Methods

Cell Samples

Bone Marrow (BM) Derived Mesenchymal Stem Cells (MSC:s)

BM MSC:s were obtained as described by Leskelä et al. (2003). Briefly, bone marrow obtained during orthopaedic surgery was cultured in Minimun Essential Alpha-Medium supplemented with 20 mM HEPES, 10% fetal calf serum, penicillin-streptomycin and 2 mM L-glutamine (all from Gibco). After a cell attachment period of 2 days the cells were washed with PBS, subcultured further by plating the cells at a density of 2000-3000 cells/cm² in the same media and replacing the medium twice a week until near confluence.

Cord Blood (CB) Derived MSC:s

Human term umbilical cord blood units were collected after delivery with informed consent of the mothers and the cord blood was processed within 24 hours of collection. Mononuclear cells (MNC:s) were isolated from each unit by Ficoll-Paque Plus (GE Healthcare Biosciences) density gradient centrifugation. The mononuclear cell fraction was plated on fibronectin (Sigma Aldrich)-coated 6-well plates (Nunc) at 10⁶ cells/well. Most of the non-adherent cells were removed as the medium was replaced the next day. The cells were cultured essentially as described for BM MSC:s above.

Both BM and CB MSCs were analyzed by flow cytometry to be negative for CD14, CD34, CD45 and HLA-DR; and positive for CD13, CD29, CD44, CD90, CD105 and HLA-ABC. The cells were shown to be able to differentiate along osteogenic, adipogenic and chondrogenic lineages.

Osteogenic Differentiation

Osteogenic differentiation of BM and CB MSC:s was induced by culturing the cells for 1-6 weeks in osteogenic induction medium: αMEM supplemented with 20 mM HEPES, 10% FCS, 2 mM glutamine, 0.1 μM dexamethasone, 10 mM β-glycerophosphate, 0.05 mM ascorbic acid-2-phosphate, and penicillin-streptomycin.

Adipogenic differentiation. To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/ml insulin, 0.1 mM indomethacin, 0.1 μM dexamethasone (Sigma-Aldrich) and penicillin-streptomycin (Gibco) before samples were prepared for glycome analysis. The medium was changed twice a week during differentiation culture.

Flow Cytometry

MSC:s were detached from culture plates by incubating with 2 mM EDTA-PBS (Versene) for 15 min at +37° C. or with 0.25% trypsin in 1 mM EDTA-PBS for 3 min at +37° C. Osteogenically differentiated MSC:s were detached by incubating with 10mM EDTA-PBS (Versene) for 30 min (cells differentiated from cord blood MSC:s) or 1 h (cells differentiated from bone marrow MSC:s) at +37° C. or with 0.25% trypsin in 1 mM EDTA-PBS for 3 min at +37° C. Cells (50 000) were incubated with primary antibodies (4 μl/100 μl diluted in 0.3% BSA-PBS) for 30 min at room temperature and washed once before incubating with secondary antibody (1:500) for 30 min at room temperature in the dark. Control cells were treated similarly but without primary antibody. Cells were analyzed with BD FACSAria (Becton Dickinson) using FITC detection at 488 nm or propidium iodide detection. Results were analyzed with BD FACSDiva software version 5.0.1 (Becton Dickinson).

Results and Discussion

Bone marrow and cord blood mesenchymal stem cells and cells differentiated from them into osteogenic and adipogenic directions were labeled by mAbs CSLEX, KM-93 and CHO-131 and analyzed by flow cytometry. The results are shown in Table 4. Each of the MAbs recognizes a different size subpopulation of each of the cell types. CHO-131 stains nearly 100% of all the different cell types studied. KM-93 stains the majority of bone marrow and cord blood mesenchymal stem cells (82% and 67%, respectively), but the staining decreases upon differentiation. CSLEX stains about 10% of bone marrow and cord blood mesenchymal stem cells and osteogenically differentiated bone marrow MSC:s. The staining of cord blood MSC:s by CSLEX increases slightly upon differentiation (to 19% for osteogenic cells and to 13% for adipogenic cells). The results show that CHO-131 recognizes an epitope that is more common in mesenchymal stem cells and cells differentiated from them, than the epitopes recognized by KM-93 and especially by CSLEX. This is in accordance with the glycan binding analysis of Example 1, which indicates that mAb CSLEX requires the sialyl Lewis x epitope for binding, but mAb CHO-131 binds to a sialylated non-fucosylated core 2 O-glycan in addition to epitopes containing sLex.

### Example 5

### O-Glycan Profile of Bone Marrow Mesenchymal Stem Cells and Hematopietic Stem Cells

Materials and Methods for Mesenchymal Stem Cells

Cell samples were prepared as described in the preceding Examples. O-glycans were detached from cellular glycoproteins by non-reductive β-elimination with saturated ammonium carbonate in concentrated ammonia at 60 ° C. essentially as described by Huang et al. (Anal. Chem. 2000, 73 (24) 6063-9) and purified by solid-phase extraction steps with C18 silica, cation exchange resin, and graphitized carbon. O-glycan profiles were analyzed by MALDI-TOF mass spectrometry separately for isolated neutral and acidic O-glycan fractions, and the result was expressed as % of total O-glycan profile for each detected O-glycan component. The purification and analysis steps were performed essentially as described in WO2007012695.

Results and Discussion

O-glycan profiling of bone marrow mesenchymal stem cells shows that the composition S2H2N2 at m/z 1329 is a major component of the O-glycome of both bone marrow mesenchymal stem cells, and cell types differentiated from them (Table 5). The composition S2H2N2 is consistent with the structure Neu5Acα2-3Galβ1-3(Neu5Acα2-3Galβ1-4GlcNAβ1-6)GalNAcα. It is, however, realized that there are other structures consistent with the same composition, but considering the common O-glycan biosynthetic routes, it is very likely that at least a part of the signal at m/z 1329 is derived from Neu5Acα2-3Galβ1-3(Neu5Acα2-3Galβ1-4GlcNAcβ1-6)GalNAcα. The abundance of the composition S2H2N2 in both mesenchymal stem cells and differentiated cell types arising from them is consistent with the observation that nearly 100% of both MSC:s and cells differentiated from them stain positive for the CHO-131 epitope. The signals corresponding to the sialyl Lewis x carrying structures, S2H2N2F1 and S1H2N2F1, are nearly nonexistent in differentiated cells. Thus the O-glycan profiles of mesenchymal stem cells and cells differentiated from them are in accordance with the proposed broader binding specificity of CHO-131, which includes, in addition to sLex-containing epitopes, the sialylated non-fucosylated core 2 O-glycan structure Neu5Acα2-3Galβ1-3(Neu5Acα2-3Galβ1-4GlcNAcβ1-6)GalNAcα.

Hematopoietic Stem Cells

O-glycan analyses of cord blood mononuclear (CB MNC), and cord blood CD133+ cells Mass spectrometric profiling of hematopoietic stem cells

The core II O-glycan (NeuNAcα3Galβ4(Fucα3)GlcNAcβ6([NeuNAcα3]Galβ3)GalNAc) was observed as molecular mass signal corresponding to disialylated structure from CD133 cells. Due to low amount of material and weak signals, the analysis was further performed from cord blood mononuclear cells (containing the stem cells and corresponding differentiated cells). The cord blood mononuclear analysis contained more material when cells are not lost during the isolation process and the core II O-glycans were observed as a major disialylated structure. Based on known O-glycan biosyunthesis, core II O-glycan is only possible human variant for such structure. The galactose residue in neutral structure was revealed to be β4-linked by specific galactosidase. B-galactosidase do not release Gal from Lewis x (Galβ4(Fucα3)GlcNAc) supporting the assignment.

The assignment of the sialylated structure core II structure was supported by presence of specific cleavage (peeling) product, wherein the 3-linked branch is elimated NeuNAcα3Galβ4(Fucα3)GlcNAcβ6GalNAc(3-aminodeoxy-GalNAc), presence of only one sialic acid further supported the assignment of the sialic acid residues of different branches of the core II O-glycan. Based on the presence of two sialic acid residues on different brancehs the fucose is likely not Fucα2 as the sialyltransferases and α2-fucosyltransferases do not normally react on the same galactose. The fucose was also not released by α2-fucosidase.

In both CB MNC samples, major fucosylated and sialylated O-glycans were observed at m/z 1184.45 (calc. m/z 1184.42 for the [NeuAc₁Hex₂HexNAc₂dHex₁-H]⁻ anion) and at m/z 1475.85 (calc. m/z 1475.52 for the [NeuAc₂Hex₂HexNAc₂dHex₁-H]⁻ anion), respectively. In CB CD133+ cells, the latter signal was observed at m/z 1475.79. Non-sialylated O-glycans of CB MNC included glycan signals at m/z 771.30 (calc. m/z 771.26 for the [Hex₂HexNAc₂+Na]⁺ ion) and m/z 917.36 (calc. m/z 917.32 for the [Hex₂HexNAc₂dHex₁+Na]⁺ ion). When further digested with S. pneumoniae β1,4-galactosidase, Hex₁HexNAc₂ signal at m/z 609.22 (calc. m/z 609.21 for [M+Na]+ ion) was increased and Hex₂HexNAc₂ signal was decreased, indicating β1,4-linked galactose; however Hex₂HexNAc₂dHex₁ was not decreased indicating non-accessible galactose.

Verification of Core II Structures by Characteristic Alkaline Peeling Product

The assignment of the sialylated structure core II structure was supported by presence of specific cleavage (peeling) product, wherein the 3-linked branch is elimated NeuNAcα3Galβ4(Fucα3)GlcNAcβ6GalNAc(a 3-aminodeaxy-GalNAc, Amine derived from ammonia/NH₄CO₃ buffer of the non-reductive beta-elimination, ref WO2007/010089), presence of only one sialic acid on the elimination fragment further supported the assignmet of the sialic acid residues of different branches of the core II O-glycan.

Cellular O-glycans were isolated by non-reducing 3-elimination (Huang et al. 2001. Anal. Chem. 73:6063-9) wherein GalNAc 6-branch fragments of mucin-type O-glycans are observed due to elimination of NeuNAca3Gal at GalNAc 3-position. Full analysis of the unusual by-product is shown with standards in WO2007/010089, page 174 line 30 to page 177 line 14, with conclusions to line 8; and fragmentation MS of deuteroacetylated glycan is shown in FIG. 12. The data indicates that the product is very characteristic to the core II O-glycans (with or without fucose).

Characteristic elimination fragments corresponding to NeuAc-Hex-HexNAc-deoxyaminoHexNAc O-glycan 6-branch fragment at m/z 899.25 (calc. m/z 899.32 for the [NeuAc₁Hex₁HexNAc₁-3-deoxyaminoHexNAc₁+Na]⁺ cation) and NeuAc-Hex-(Fuc-)HexNAc-3-deoxyaminoHexNAc O-glycan 6-branch fragment at m/z 1045.29 (calc. m/z 1045.38 for the [NeuAc₁Hex₁dHex₁HexNAc₁deoxyamino-HexNAc₁+Na]⁺ cation) were observed in CB MNC.

The CHO-131 antibody shows the distribution of the core II slex among the cord blood cells showing enrichment to the stem cell fraction.

TABLE 1
Glycan binding specificity of mAb CHO-131. 50 best binders out of the 442 glycan structures on the microarray are shown.

| Chart Number | Structure | RFU | STDEV |
|---|---|---|---|
| 220 | Neu5Aca2-3(6-O-Su)Galb1-4(Fuca1-3)GlcNAcb-Sp8 | 36255 | 2029 |
| 244 | Neu5Aca2-3Galb1-4(Fuca1-3)GlcNAcb-Sp8 | 34336 | 424 |
| 245 | Neu5Aca2-3Galb1-4(Fuca1-3)GlcNAcb1-3Galb-Sp8 | 33335 | 2568 |
| 332 | (Neu5Aca2-3-Galb1-3)(((Neu5Aca2-3-Galb1-4(Fuca1-3))GlcNAcb1-6)GalNAc-Sp14 | 33035 | 1257 |
| 290 | Galb1-3(Neu5Aca2-3Galb1-4(Fuca1-3)GlcNAcb1-6)GalNAca-Sp14 | 32407 | 1741 |
| 242 | Neu5Aca2-3Galb1-4(Fuca1-3)GlcNAcb1-3Galb1-4(Fuca1-3)GlcNAcb1-3Galb1-4(Fuca1-3) | 29505 | 2325 |
| | GlcNAcb-Sp0 | | |
| 246 | Neu5Aca2-3Galb1-4(Fuca1-3)GlcNAcb1-3Galb1-4GlcNAcb-Sp8 | 24587 | 1339 |
| 310 | Neu5Aca2-3Galb1-3(Neu5Aca2-3Galb1-4GlcNAcb1-6)GalNAca-Sp14 | 23122 | 1466 |
| 43 | Neu5Aca2-3[6OSO3]Galb1-4GlcNAcb-Sp8 | 22706 | 2924 |
| 243 | Neu5Aca2-3Galb1-4(Fuca1-3)GlcNAcb-Sp0 | 20068 | 13427 |
| 271 | Neu5Gca2-3Galb1-4(Fuca1-3)GlcNAcb-Sp0 | 9521 | 911 |
| 277 | Galb1-3(Neu5Aca2-3Galb1-4GlcNAcb1-6)GalNAca-Sp14 | 6497 | 722 |
| 299 | GlcAb1-3GlcNAcb-Sp8 | 5434 | 1262 |
| 440 | [6OSO3]Galb1-3[6OSO3]GlcNAc-Sp0 | 1773 | 170 |
| 287 | [6OSO3]Galb1-4[6OSO3]GlcNAcb-Sp0 | 1306 | 44 |
| 213 | Fuca1-2[6OSO3]Galb1-4[6OSO3]Glc-Sp0 | 1100 | 182 |
| 25 | [3OSO3]Galb1-4[6OSO3]Glcb-Sp8 | 903 | 111 |
| 337 | GlcNAca1-4Galb1-4GlcNAcb1-3Galb1-4(Fuca1-3)GlcNAcb1-3Galb1-4(Fuca1-3)GlcNAcb-Sp0 | 706 | 340 |
| 346 | Galb1-4GlcNAcb1-2Mana1-3(Mana1-6)Manb1-4GlcNAcb1-4GlcNAcb-Sp12 | 639 | 431 |
| 32 | [3OSO3]Galb1-4[6OSO3]GlcNAcb-Sp8 | 584 | 168 |
| 42 | [6OSO3]Galb1-4[6OSO3]Glcb-Sp8 | 505 | 208 |
| 322 | Fuca1-3(Galb1-4)GlcNAcb1-2Mana1-3(Fuca1-3(Galb1-4)GlcNAcb1-2Mana1-6) | 478 | 802 |
| | Manb1-4GlcNAcb1-4GlcNAcb-Sp20 | | |
| 374 | Neu5Aca2-3Galb1-4(Fuca1-3)GlcNAcb1-3GalNAca-Sp14 | 451 | 71 |
| 36 | [4OSO3][6OSO3]Galb1-4GlcNAcb-Sp0 | 427 | 125 |
| 210 | [3OSO3]Galb1-4(Fuca1-3)[6OSO3]GlcNAc-Sp8 | 403 | 114 |
| 9 | Neu5Aca-Sp11 | 383 | 120 |
| 358 | Fuca1-2Galb1-4(Fuca1-3)GlcNAcb1-2Mana1-3(Fuca1-2Galb1-4(Fuca1-3)GlcNAcb1-2Mana1-6) | 346 | 135 |
| | Manb1-4GlcNAcb1-4GlcNAb-Sp20 | | |
| 35 | [3OSO3]Galb-Sp8 | 345 | 67 |
| 191 | G-ol-Sp8 | 304 | 258 |
| 193 | GlcAb-Sp8 | 274 | 63 |
| 323 | Neu5Ac(9Ac)a2-3Galb1-4GlcNAcb-Sp0 | 271 | 262 |
| 31 | [3OSO3]Galb1-4[6OSO3]GlcNAcb-Sp0 | 266 | 40 |
| 241 | Neu5Aca2-3Galb1-4(Fuca1-3)[6OSO3]GlcNAcb-Sp8 | 256 | 89 |
| 256 | Neu5Aca2-6Galb1-4[6OSO3]GlcNAcb-Sp8 | 243 | 70 |
| 53 | Neu5Aca2-6Galb1-4GlcNAcb1-2Mana1-3(Neu5Aca2-6Galb1-4GlcNAcb1-2Mana1-6) | 240 | 158 |
| | Manb1-4GlcNAcb1-4GlcNAcb-Sp12 | | |
| 390 | Gala1-3Galb1-3GlcNAcb1-2Mana1-3(Gala1-3Galb1-3GlcNAcb1-2Mana1-6) | 232 | 241 |
| | Manb1-4GlcNAcb1-4GlcNAc-Sp19 | | |
| 192 | GlcAa-Sp8 | 227 | 41 |
| 339 | GlcNAca1-4Galb1-3GalNAc-Sp14 | 209 | 195 |
| 55 | Fuca1-2Galb1-3GalNAcb1-3Gala-Sp9 | 190 | 57 |
| 311 | Neu5Aca2-3Galb1-3(Neu5Aca2-6)GalNAca-Sp14 | 182 | 147 |
| 424 | Gala1-3(Fuca1-2)Galb1-4GlcNAcb1-2Mana1-3(Gala1-3(Fuca1-2)Galb1-4GlcNAcb1-2Mana1-6) | 179 | 138 |
| | Manb1-4GlcNAcb1-4(Fuca1-6)GlcNAcb-Sp22 | | |
| 321 | Neu5Aca2-6Galb1-4GlcNAcb1-2Mana1-3(Neu5Aca2-3Galb1-4GlcNAcb1-2Mana1-6) | 175 | 293 |
| | Manb1-4GlcNAcb1-4GlcNAcb-Sp12 | | |
| 297 | GalNAca-Sp15 | 170 | 134 |
| 415 | GalNAca1-3(Fuca1-2)Galb1-4(Fuca1-3)GlcNAcb1-3GalNAc-Sp14 | 165 | 159 |
| 288 | 6-H2PO3Glcb-Sp10 | 159 | 239 |
| 146 | Galb1-4[6OSO3]Glcb-Sp0 | 148 | 52 |
| 348 | Galb1-4GlcNAcb1-2Mana1-3(Galb1-4GlcNAcb1-2Mana1-6)Manb1-4GlcNAcb1-4(Fuca1-6) | 142 | 114 |
| | GlcNAcb-Sp22 | | |
| 382 | Galb1-3GlcNAcb1-3(Galb1-3GlcNAcb1-3Galb1-4(Fuca1-3)GlcNAcb1-6)Galb1-4Glc-Sp21 | 140 | 92 |
| 281 | Galb1-4(Fuca1-3)[6OSO3]Glc-Sp0 | 138 | 26 |
| 351 | [6OSO3]GlcNAcb1-3Gal b1-4GlcNAc-b-Sp0 | 138 | 129 |

TABLE 2
Glycan binding specificity of mAb CSLEX. 50 best binders out of the 442 glycan structures on the microarray are shown.

| Chart Number | Structure | RFU | STDEV |
|---|---|---|---|
| 242 | Neu5Aca2-3Galb1-4(Fuca1-3)GlcNAcb1-3Galb1-4(Fuca1-3)GlcNAcb1-3Galb1-4(Fuca1-3) | 31104 | 6242 |
| | GlcNAcb-Sp0 | | |
| 246 | Neu5Aca2-3Galb1-4(Fuca1-3)GlcNAcb1-3Galb1-4GlcNAcb-Sp8 | 27193 | 1600 |
| 245 | Neu5Aca2-3Galb1-4(Fuca1-3)GlcNAcb1-3Galb-Sp8 | 22997 | 1245 |
| 220 | Neu5Aca2-3(6-O-Su)Galb1-4(Fuca1-3)GlcNAcb-Sp8 | 22795 | 3485 |
| 244 | Neu5Aca2-3Galb1-4(Fuca1-3)GlcNAcb-Sp8 | 19491 | 2468 |
| 332 | (Neu5Aca2-3-Galb1-3)(((Neu5Aca2-3-Galb1-4(Fuca1-3))GlcNAcb1-6)GalNAc-Sp14 | 19162 | 1919 |
| 243 | Neu5Aca2-3Galb1-4(Fuca1-3)GlcNAcb-Sp0 | 13988 | 737 |
| 43 | Neu5Aca2-3[6OSO3]Galb1-4GlcNAcb-Sp8 | 12653 | 1722 |
| 271 | Neu5Gca2-3Galb1-4(Fuca1-3)GlcNAcb-Sp0 | 4766 | 987 |
| 379 | Galb1-3GlcNAcb1-3(Galb1-4(Fuca1-3)GlcNAcb1-6)Galb1-4Glc-Sp21 | 712 | 1383 |
| 257 | Neu5Aca2-6Galb1-4GlcNAcb-Sp0 | 424 | 825 |
| 54 | Neu5Aca2-6Galb1-4GlcNAcb1-2Mana1-3(Neu5Aca2-6Galb1-4GlcNAcb1-2Mana1-6) | 245 | 465 |
| | Manb1-4GlcNAcb1-4GlcNAcb-Sp13 | | |
| 374 | Neu5Aca2-3Galb1-4(Fuca1-3)GlcNAcb1-3GalNAca-Sp14 | 112 | 109 |
| 171 | GlcNAcb1-3Galb-Sp8 | 106 | 172 |
| 290 | Galb1-3(Neu5Aca2-3Galb1-4(Fuca1-3)GlcNAcb1-6)GalNAca-Sp14 | 95 | 129 |
| 407 | Neu5Aca2-6Galb1-3GlcNAcb1-3(Galb1-4GlcNAcb1-6)Galb1-4Glc-Sp21 | 95 | 133 |
| 346 | Galb1-4GlcNAcb1-2Mana1-3(Mana1-6)Manb1-4GlcNAcb1-4GlcNAcb-Sp12 | 64 | 31 |
| 136 | Galb1-3GalNAcb1-4Galb1-4Glcb-Sp8 | 58 | 57 |
| 91 | GalNAcb1-3GalNAca-Sp8 | 56 | 33 |
| 215 | Neu5Aca2-8Neu5Aca2-8Neu5Aca2-8Neu5Aca2-3(GalNAcb1-4)Galb1-4Glcb-Sp0 | 53 | 29 |
| 291 | Galb1-3Galb1-4GlcNAcb-Sp8 | 46 | 60 |
| 261 | Neu5Aca2-6Galb1-4Glcb-Sp0 | 45 | 46 |
| 315 | Neu5Aca2-6Galb1-4GlcNAcb1-2Mana1-3(GlcNAcb1-2Mana1-6)Manb1-4GlcNAcb1-4GlcNAcb-Sp12 | 45 | 16 |
| 326 | Neu5Aca2-3Galb1-3(Fuca1-4)GlcNAcb1-3Galb1-3(Fuca1-4)GlcNAcb-Sp0 | 42 | 7 |
| 350 | Galb1-3(Fuca1-4)GlcNAcb1-2Mana1-3(Galb1-3(Fuca1-4)GlcNAcb1-2Mana1-6) | 42 | 24 |
| | Manb1-4GlcNAcb1-4GlcNAcb-Sp19 | | |
| 339 | GlcNAca1-4Galb1-3GalNAc-Sp14 | 41 | 36 |
| 337 | GlcNAca1-4Galb1-4GlcNAcb1-3Galb1-4(Fuca1-3)GlcNAcb1-3Galb1-4(Fuca1-3)GlcNAcb-Sp0 | 41 | 13 |
| 191 | G-ol-Sp8 | 40 | 19 |
| 419 | Fuca1-2Galb1-3GlcNAcb1-3GalNAc-Sp14 | 39 | 17 |
| 299 | GlcAb1-3GlcNAcb-Sp8 | 39 | 10 |
| 307 | Mana1-6(Mana1-3)Mana1-6(Mana1-3)Manb-Sp10 | 37 | 27 |
| 137 | Galb1-3Galb-Sp8 | 37 | 26 |
| 311 | Neu5Aca2-3Galb1-3(Neu5Aca2-6)GalNAca-Sp14 | 36 | 22 |
| 202 | Mana1-2Mana1-6(Mana1-3)Mana1-6[Mana1-2Mana1-2Mana1-3]Manb1-4GlcNAcb1-4GlcNAcb-Sp12 | 35 | 25 |
| 161 | Galb1-4GlcNAcb-Sp8 | 34 | 33 |
| 211 | Fuca1-2[6OSO3]Galb1-4GlcNAc-Sp0 | 33 | 22 |
| 387 | Galb1-3GlcNAcb1-3GalNAca-Sp14 | 33 | 30 |
| 178 | GlcNAcb1-4Galb1-4GlcNAcb-Sp8 | 33 | 25 |
| 295 | Galb1-4GlcNAca1-6Galb1-4GlcNAcb-Sp0 | 30 | 19 |
| 82 | GalNAca1-3(Fuca1-2)Galb1-4GlcNAcb-Sp0 | 30 | 23 |
| 155 | Galb1-4GlcNAcb1-3Galb1-4Glcb-Sp0 | 29 | 19 |
| 287 | [6OSO3]Galb1-4[6OSO3]GlcNAcb-Sp0 | 29 | 22 |
| 87 | GalNAca1-3(Fuca1-2)Galb-Sp18 | 29 | 13 |
| 177 | GlcNAcb1-4(GlcNAcb1-6)GalNAca-Sp8 | 29 | 14 |
| 266 | Galb1-3(Fuca1-4)GlcNAcb1-3Galb1-3(Fuca1-4)GlcNAcb-Sp0 | 29 | 37 |
| 267 | Neu5Acb2-6GalNAca-Sp8 | 29 | 28 |
| 321 | Neu5Aca2-6Galb1-4GlcNAcb1-2Mana1-3(Neu5Aca2-3Galb1-4GlcNAcb1-2Mana1-6) | 28 | 20 |
| | Manb1-4GlcNAcb1-4GlcNAcb-Sp12 | | |
| 144 | Galb1-4(Fuca1-3)GlcNAcb1-4Galb1-4(Fuca1-3)GlcNAcb-Sp0 | 28 | 29 |
| 190 | Glcb1-6Glcb-Sp8 | 28 | 33 |
| 135 | Galb1-3GalNAcb1-4(Neu5Aca2-3)Galb1-4Glcb-Sp0 | 28 | 40 |

TABLE 3
Binding of anti-sLex and anti-Lex antibodies to enzymatically
α1,3-fucosylated bone marrow mesenchymal stem cells and
control cells incubated with reaction buffer without enzyme. The
binding of the antibodies was analyzed by flow cytometry and the
results are expressed as the percentage of cells that stains positive.

| | CHO-131 | CSLEX | HECA 452 | TG-1 |
|---|---|---|---|---|
| BMMSC cell sample | % pos | | | |
| buffer control | 52 | 3.8 | 0.8 | 2.1 |
| a1,3-fucosylation | 99.7 | 97.4 | 72.6 | 37.2 |

TABLE 4
FACS analysis of labeling of bone marrow (BM) and cord blood (CB)
mesenchymal stem cells (MSC) and cells differentiated from them into
osteogenic (OG) and adipogenic (AD) directions by mAbs CSLEX,
KM-93 and CHO-131. The results are shown as percentages of cells that
stain positive for the antibodies. Standard deviations are given when more
than one experiment has been performed.

| | BM-MSC | BM-OG | CB-MSC | CB-OG | CB-AD |
|---|---|---|---|---|---|
| | % pos | % pos | % pos | % pos | % pos |
| mAb Clone | cells | cells | cells | cells | cells |
| CSLEX | 8.5 ± 13.5 | 10.4 | 7.8 ± 5.9 | 19.0 | 13.5 |
| KM-93 | 82.1 | 55.7 ± 9.4 | 67.5 ± 4.6 | 12.6 | 49.1 |
| CHO-131 | 90.8 ± 11.5 | 97.5 | 99.7 ± 0.12 | 98.6 | 99.9 |

TABLE 5

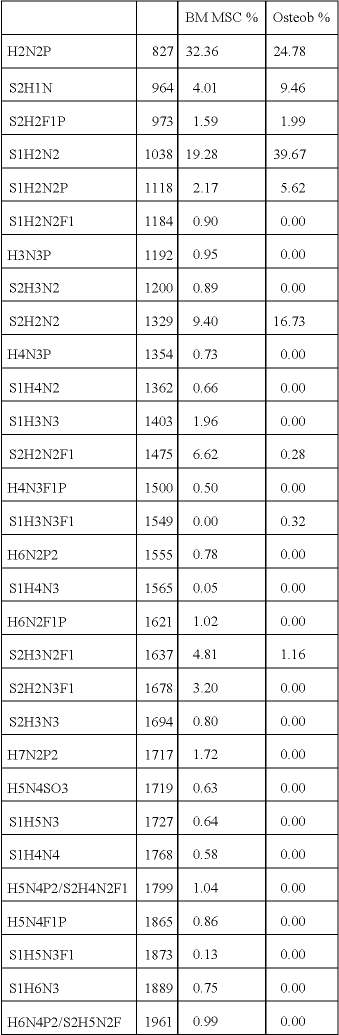

Acidic N-glycan profile of bone marrow mesenchymal stem cells (BM MSC) and osteoblasts differentiated from them. The O-glycan profiles were analyzed by MALDI-TOF mass spectrometry, and the result is expressed as % of total O-glycan profile for each detected O-glycan component. The composition S2H2N2, which is consistent with the structure Neu5Acα2-3Galβ1-3(Neu5Acα2-3Galβ1-4GlcNAcβ1-6)GalNAcα is highlighted.

1. A complex of an antibody and an isolated glycan comprising a target structure according to Formula 1
Neu5Acα2-3Galβ1-3[Neu5Acα2-3Galβ1-4(Fucα1-3)ₘGlcNAcβ1-6]GalNAc[α]ₙ,
wherein m and n are integers 0 or 1, and the larger reducing end derivatives and conjugates thereof, and wherein said antibody is also capable of binding structure
Neu5Acα2-3Galβ1-4(Fucα1-3)GlcNAcβ1, and/or
a structure according to Formula 1b
Galβ1-3[Neu5Acα2-3Galβ1-4(Fucα1-3)ₘGlcNAcβ1-6]GalNAc[α]ₙ,
wherein m and n are integers 0 or 1.

2. The complex according to claim 1, wherein said antibody is bound to the structure according to Formula 1, when m is 1.

3. The complex according to claim 2, wherein said antibody is also capable of binding the structure according to Formula 1, when m is 0.

4. The complex according to claim 2, wherein said antibody is also capable of binding the structure according to Formula 1b, when m is 1, but is not essentially capable of binding to the structure according to Formula 1b, when m is 0.

5. The complex according to claim 2, wherein said antibody is not essentially capable of binding to the structure according to Formula 1, when m is 0.

6. The complex according to claim 5, wherein said antibody is not capable of binding to the structure(s) according to Formula 1b.

7. The complex according to claim 1, wherein said complex is in an array of glycan structures, and optionally the array comprises said glycan the antibody is capable of binding to and optionally further said saccharides the antibody is not capable of binding to.

8. The complex according to claim 7, wherein said glycan array is a solid phase conjugated saccharide array.

9. A method of using the complex and/or target saccharides according to claim 1, in an assay including a solid phase assay or liquid phase assay for the screening of antibodies with diasialyl core II or CHO-131-type -specificity.

10. The method according to claim 9 comprising a step of contacting a sample containing antibodies with a disialyl saccharide according to Formula I.

11. The method according to claim 10 wherein the method includes steps of
i) providing a sample comprising at least one antibody or functional antibody fragment binding to an antigen;
ii) contacting the sample with a glycan structure comprising terminal non-reducing end target structure according to the formula I; and measuring the binding of the antibody to the oligosaccharide sequence.
iii) optionally contacting the antibody sample with at least one control glycan structure;
iv) optionally selecting antibody with specific binding to the target structures but low or non-existent binding to specificity control saccharides, or in a specific embodiment selecting antibodies with additionally or specifically corresponding sLex specificity;
v) optionally using an oligosaccharide sequence comprising the terminal non-reducing end target saccharide sequence or being the target oligosaccharide or glycopeptide for the inhibition of the binding of the antibody the oligosaccharide sequence; and
vi) optionally using enzyme selected from the group of fucosyltransferase, sialyltransferase or α3-sialidase enzymes to optimize or reduce the amount of the antibody target structures on cells.

12. The method according to claim 11, for production of non-fucosyl variants of disialyl core II or CHO131 type-antibodies wherein m is 1 and n is 0 or 1.

13. The method according to claim 10 for optimization of the binding activity of a CHO131-type antibody using the disialyl core II oligosaccharide sequence.

14. The method according to claim 9, involving the use of the target glycan comprising control material and/or the complex of claim 1 for validation of the analysis of the antibody binding to cells or other biological materials.

15. The method according to claim 14, wherein said control material is a purified oligosaccharide or synthetic chemical conjugate thereof.

16. The method according to claim 15, wherein the target epitope is conjugated to a solid surface or to control cells in a solid phase assay or used as a soluble inhibitor or soluble analyst (e.g. labelled conjugate for a fluorescence polarization assay) to validate the binding specificity of the antibody.

17. A disialyl core II or CHO131-antibody analysis kit comprising the target saccharide sequence according to Formula I comprising glycan or glycoconjugate or a cell sample optimized with the glycan structure expression, for the validation of the analysis of CHO131 antibody target-structure in cells or tissues

18. The method according to claim 9, involving use of a specific fucosyltransferase and/or sialyltransferase enzyme for optimizing the presence of the CHO target structures according to Formula I on cell surface and/or use of an α3-sialidase to reduce the amount of the structure on cells.

19. The method according to claim 9 involving further use of the antibodies produced or validated by the method for the analysis of stem cells or cancer cells or other cells or tissues known to bind to CHO-antibodies including human embryonic type stem cells, hematopoietic stem cells, mesenchymal stem cells and osteogenically or adipocyte differentiated mesenchymal stem cells.
